(19) United States
(12) Patent Application Publication
(10) Pub. No.: US 2016/0367347 A1
(43) Pub. Date: Dec. 22, 2016

(54) LIGHT CURING DEVICE

(71) Applicant: Ivoclar Vivadent AG, Schaan (LI)

(72) Inventors: Bruno Senn, Buchs (CH); Dario Tommasini, Mastrils (CH)

(21) Appl. No.: 15/181,776

(22) Filed: Jun. 14, 2016

(30) Foreign Application Priority Data
Jun. 17, 2015 (EP) 15172551.2

(51) Int. Cl. A61C 13/15; A61C 1/00; A61B 1/247

(52) U.S. Cl. CPC A61C 19/004; A61B 1/247; A61C 1/0015; A61B 2090/306

(57) ABSTRACT

A light curing device for curing dental materials, having a handpiece (14) and a base station (12), wherein the handpiece (14) includes a light source, a light-conducting rod (22), which is attached to the handpiece (14) in particular in a replaceable and detachable manner, and at least one sensor (30) which is positioned in the handpiece (14), wherein the light curing device (10) includes a control device (18) with at least one curing program for polymerizing the dental material. It is characterized in that a mirror (28) with a predetermined reflection parameter with a reflection value of more than 90% is provided, in particular at the base station (12) of the light curing device (10) or at the handpiece (14) of the light curing device (10), and in that the control device (18) includes a calibration program which can be actuated automatically or by user intervention when the light curing device (10) with the front end (24) of the light-conducting rod (22) is directed towards the mirror (28).

### CROSS-REFERENCE TO RELATED APPLICATIONS

This application claims priority to European Patent Application No. 15172551.2 filed on Jun. 17, 2015, the disclosure of which is incorporated herein by reference in its entirety.

### TECHNICAL FIELD

The invention relates to a light curing device for dental materials.

### BACKGROUND OF THE INVENTION

It has been known for a long time that for ensuring a reliable polymerization or curing process of dental materials it is decisive to provide an exactly predetermined amount of light. The target amount of light depends on many factors, for instance on the shape and the layer thickness as well as on the mass of the dental restoration to be cured, the photo initiator which triggers the polymerization process, the wavelength range emitted by the light curing device to its light-conducting rod, but indirectly also on, for instance, the light source itself which can be subjected to degradation, or also on the quality of the light-conducting rod which can, for instance, be broken—without the user having noticed any cracks.

On the other hand, it has been known for a long time that it is recommended to calibrate the light source to homogenize the output of light. As one of numerous examples hereof it is referred to DE 196 36 266 A1 and corresponding U.S. Pat. No. 5,912,470, which is hereby incorporated by reference, according to which the light source thereat is to be controlled by a detecting device for measuring the light intensity in the device (see column 3, line 48 et seq. of DE 196 36 266 A1).

Here, it is disadvantageous that the light-conducting rod cannot be involved in the calibration process.

However, improved solutions have also become known in this respect which involve the light-conducting rod. For this purpose, it is referred to DE 10 2007 052 643 A1 and corresponding US 20090114844, which is hereby incorporated by reference, which comprises a light sensor at the base station. In this solution, the handpiece must be directed at the sensor area with the end of the light-conducting rod, and the light emission is to be measured in this way, in particular also the light intensity.

If several—equal—light curing devices are used, there is the possibility that base station and handpiece are not correctly allocated by the dentist, and that, as a result, the measurement of the emitted light intensity refers to the wrong light curing device. This holds particularly true if the calibration information is only transmitted if the light curing device is inserted, as is the case with the solution according to DE 10 2007 052 643 A1 and corresponding US 20090114844, which is hereby incorporated by reference, in which the measured light power has to be stored temporarily in the base station.

Further, an even more reliable detection of a micro-fracture in the light-conducting rod would be desirable.

### SUMMARY OF THE INVENTION

Thus, the invention is based on the task of providing a light curing device for curing dental materials according to the preamble of claim 1 which is further improved with regard to the detection capacity and reliability, wherein the costs for the realization thereof should at most be within the range of the solutions which have already been known.

This task is inventively solved by claim 1. Advantageous developments may be taken from the subclaims.

Surprisingly, the inventive solution of using a low-priced mirror whose reflected light is led through the light-conducting rod again considerably improves the detectability of micro-cracks. The micro-cracks regularly scatter the introduced light and increase the damping by the light-conducting rod in this respect; by the inventive doubling of the damping with respect to the proper operation of the light-conducting rod in the calibration process the rate of detection can be improved considerably.

As the control device is directly allocated to the handpiece a confusion or an incorrect allocation of the detected data is impossible; surprisingly, this problem which is present in the prior art can be avoided securely by means of decidedly simple measures and accordingly cheaply, in this way.

According to the invention, the incidence of light onto the sensor which is located inside the handpiece can trigger a calibration program. For this purpose, it is preferable that a threshold value is set in order to avoid that the calibration program is started already due to reflection at, for instance, a tooth surface.

Alternatively, it is also possible to start the calibration program by operating a micro-switch which is actuated when the end of the light-conducting rod is aligned in a predetermined position relative to the inventive mirror. A third possibility is to trigger the calibration program when the user wishes it to be triggered.

According to the invention, it is favorable that the mirror is disposed at or at least close to the handpiece. The arrangement at the handpiece can, for instance, be realized by means of a mirror attachment at the front end of the light-conducting rod. In the “standard operation” it is pivoted away and is preferably in comparatively close contact with the light-conducting rod. During the calibration operation, it is pivoted to the front and extends with the mirror surface parallel to the output end of the light-conducting rod, just before it, such that the emitted radiation is reflected substantially completely with the reflection efficiency of the mirror.

It is also possible to provide the mirror attachment of the light-conducting rod with a sliding sleeve in addition to the pivotability such that the attached sleeve is pushed back to the root of the light-conducting rod and such that then the region of the light-conducting rod which is introduced into the mouth is free of additional elements.

In this connection, it is favorable that the attachment does not hinder the autoclavability of the pluggable light-conducting rod.

Alternatively, the mirror can be attached to the base station. There, it is preferable that an alignment aid is realized just like in case of the attached sleeve which ensures that the mirror extends exactly perpendicular relative to the light-emitting surface of the light-conducting rod in the calibration mode. The alignment aid can be realized, for instance, by means of a sleeve into which the front end of the light-conducting rod can be inserted and which holds this front end at an angle of 90°, i.e. perpendicular to the mirror. This embodiment also ensures that the same calibration conditions are always present in the calibration mode.

The sensor is disposed in the handpiece such that it detects the reflected radiation which passes through the light-conducting rod again. Preferably, it is attached in an optically decoupled manner from the light source, for instance in the beam path thereof behind it, and it receives the reflected radiation of the mirror by means of optical coupling elements after it has passed through the light-conducting rod.

In an advantageous embodiment it is provided that the quality and current power of the light curing device in the calibration mode is displayed to the user of the light curing device appropriately, for instance by means of color indications such as red, yellow, green or as a numeric value which indicates the light power. Thus, the user can determine a degradation of a light curing device at an early stage and can, for instance, ensure that dirt does not affect the light power.

It is also possible to invite the user to clean or exchange the light guide by means of a corresponding display.

Basically, pluggable light guides are preferred as they are autoclavable. However, they involve the danger that the lens system of the light curing device gets dirty, for instance dusty, when the light-conducting rod is not inserted again accidentally after it has been cleaned. This also results in a degradation of the light power, wherein it is possible, however, that the user performs cleaning himself/herself according to the manual of the light curing device, for instance using a soft brush or the like to clean the optics.

The light-conducting rod itself can have deposits in particular at its front end which is often in contact with the mouth of the patient, which deposits cannot be removed by autoclaving or not completely. Here, it can be recommended to the user to clean this front end via a display, such that an early replacement of the light-conducting rod which is operable apart from that can be avoided.

On the other hand, light-conducting rods can have micro-cracks, i.e. cracks which cannot be detected from the outside without further ado—unless the typically black plastic protection cover is removed. In this state, the light-conducting rod is typically not broken completely, but only comprises cracks which have been caused, for instance, by an unclean handling, by dropping the device or the like.

These micro-cracks lead to an increase in the damping of the permeability of the light-conducting rod. With a typical crack, the light power is reduced to the factor of 0.8 in operation. According to the invention, however, the significance of this degradation is increased considerably in the calibration mode; the reflected radiation is again reduced by the factor of 0.8 such that altogether there is a reduction by 0.8 times 0.8, i.e. 0.64, which can be detected considerably more easily by the sensor and the related control device of the light curing device.

According to the invention, it is provided that in the calibration mode discontinuous light is emitted by the light source, for instance sinusoidal or pulsed light. This makes possible to differentiate between ambient light and the light emitted by the light source. In this connection, the control device evaluates the amplitude of the received output signal of the sensor without further ado and uses it as useful light for the calibration process.

Within the control range a self-recalibration can also be realized in the calibration mode; for instance, an increase or a decrease of the light power by plus/minus 20% can be predefined.

According to the invention, it is also favorable if the handpiece of the light curing device detects the removal of the light guide automatically and then immediately requests the calibration mode or requests to have it performed the next time the device is turned on.

This enables the automatic compensation of series tolerances of light-conducting rods when exchanging the light-conducting rods. Additionally, the fact that the permeability is improved in most cases when the light-conducting rod has been cleaned is taken into account such that a calibration is recommended in this respect.

Preferably, the sensor(s) is/are arranged slightly outside of the central axis of the rear end of the light-conducting rod. Thus, they preferably detect the reflected light transmitted on the outer circumference of the light-conducting rod, while the light source preferably impinges on the central area of the light-conducting rod.

### BRIEF DESCRIPTION OF THE DRAWINGS

Further advantages, details and features may be taken from the following description of an exemplary embodiment of the invention in conjunction with the drawings, in which:

FIG. 1 shows a schematic view of an embodiment of an inventive light curing device in a perspective illustration;

FIG. 2 shows the plan view of the embodiment according to FIG. 1; and

FIG. 3 shows a slightly schematized view of a further embodiment of an inventive light curing device.

FIG. 4 shows a sectional view of a portion of FIG. 3.

### DETAILED DESCRIPTION

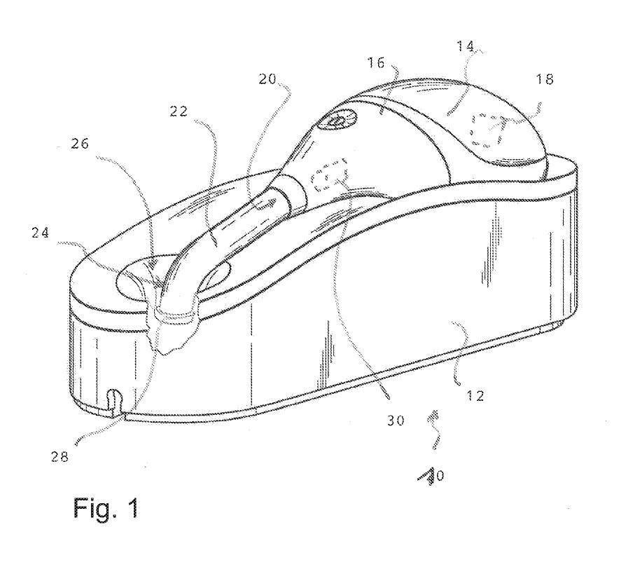

The light curing device 10 illustrated in FIG. 1 comprises a base station 12 and a handpiece 14 which can be separated from one another in a way known per se. In the illustrated exemplary embodiment, the base station 12 provides a spatially fixed supporting or receiving element for the handpiece 14, and furthermore the possibility to automatically charge the accumulator of the handpiece 14.

Furthermore, a preferably wireless data transfer between the handpiece 14 and the base station 12 is provided. It serves, for instance, to adjust the charging current to the requirements. Bidirectional communication is also possible optionally, for instance, when data about the operation of the handpiece in the dental practice which may, for instance, have available several inventive light curing devices is to be transferred to a central station.

The handpiece 14 comprises a housing 16 which receives a schematically illustrated control device 18. At its front end the housing 16 comprises a reception 20 for a light-conducting rod 22. In the exemplary embodiment illustrated, the light-conducting rod 22 is replaceable and snaps into place in the reception 20; in another embodiment it is connected fixedly to the housing 16.

In a way known per se, the light-conducting rod 22 is cranked off, in fact before its front end 24. In the resting position illustrated the end rests in a recess 26 of the base station 12, wherein the light-emitting end of the light-conducting rod 22 protrudes into the recess 26.

According to the invention, the base station 12 comprises a mirror 28 at the bottom of the recess 26 which exactly fits the front end 24 of the light-conducting rod 22 and which is exactly aligned relative to it when the handpiece 14 is in the base station 12 in the resting position.

The mirror is aligned slightly obliquely relative to the horizontal according to the light-emitting surface of the light-conducting rod, and comprises a reflection value of more than 95% in the clean state. In this way, the light leaving the light-conducting rod 22 is reflected almost completely and returned into the light-conducting rod 22.

Adjacent to the rear end of the light-conducting rod 22 and to the reception 20 a sensor 30 is provided in the handpiece 14 which detects the light which is returned by the mirror 28 and again led through the light-conducting rod 22 and whose output signal is supplied to the control device 18.

Now, the control device 18 comprises a specific calibration program which triggers a calibration program upon a certain incidence of light onto the sensor 30 or alternatively upon mechanical activation by the dentist using the device. The calibration program regulates a rated light power of the light source of the handpiece 14 which is also adjacent to the reception 20 to a rated value such that the light output always takes place in the same manner in operation.

In a further alternative embodiment, it is provided that the calibration program is automatically started every time the handpiece 14 is inserted into the base station 12. Adapting the light power to the target value will then take place automatically within the control reserve.

When the control reserve of the light power is almost used up, a message to the dentist or to another user is preferably sent by means of which he/she is prompted to clean or replace the light-conducting rod.

It is to be understood that the inventive solution of a light curing device with an integrated mirror is not limited to pin-shaped handpieces; rather, widely-used pistol-shaped light curing devices can be equipped in the same manner according to the invention.

The mirror 28 does not have to be located at the position either at which the handpiece is in the resting position; however, this is preferred. Alternatively, for instance, a recess 26 can be provided at the side of the base station 12 which is equipped with the mirror 28 and into which the front end 24 of the light-conducting rod 22 can be inserted.

This recess 26 will then offer an alignment aid for aligning the light curing device relative to the mirror 28, just like the recess 26 according to FIG. 1 offers an alignment aid in this respect.

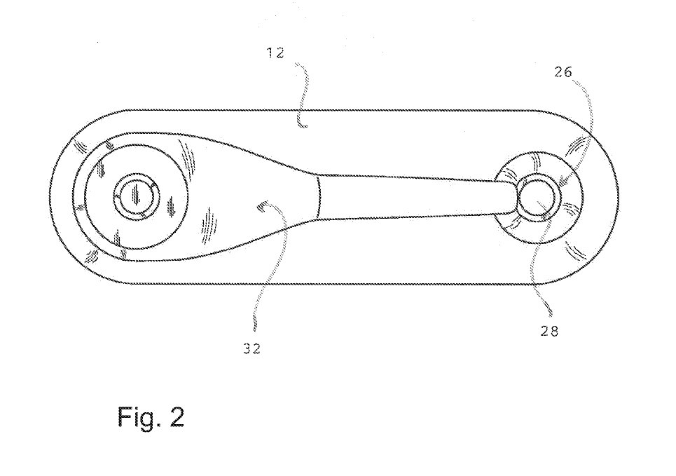

FIG. 2 illustrates the base station 12 in the plan view. The base station 12 comprises quasi a type of charging tray 32 for the handpiece 14. The exposed mirror 28 is apparent which is attached to the bottom of the recess 26. As the recess 26 is rounded the mirror can be kept clean relatively easily, and the spatial fixation, that is to say the alignment aid, takes place between the outer contour of the handpiece 14 and the charging tray 32 of the base station 12.

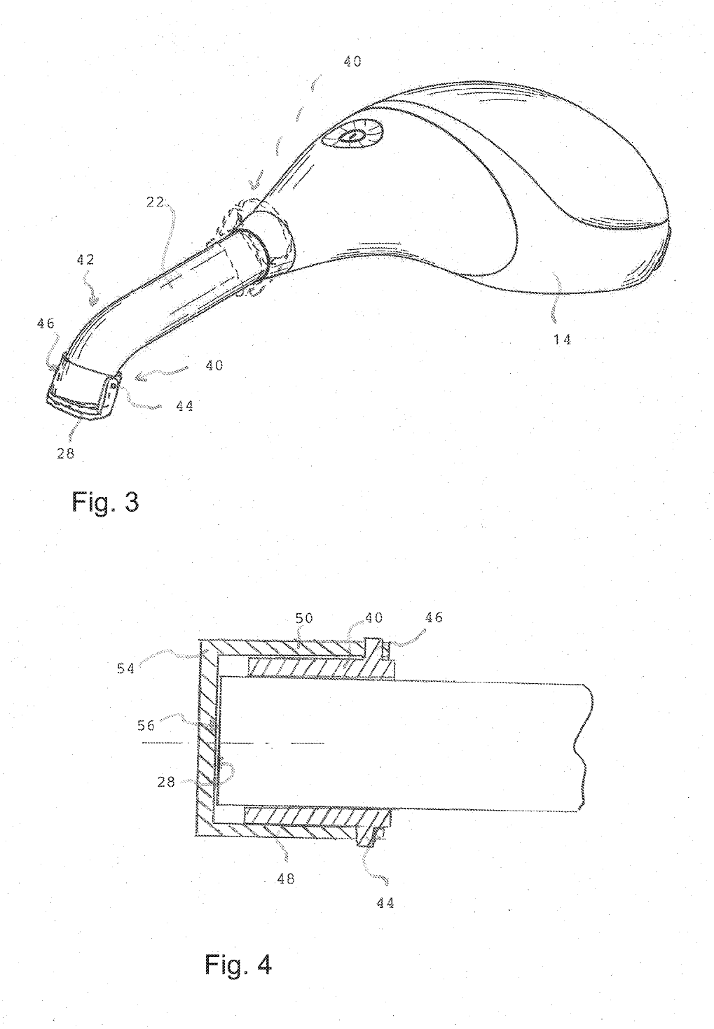

FIG. 3 shows a modified embodiment of the inventive light curing device. In this embodiment the mirror 28 is attached to the handpiece 14 itself. A sliding sleeve 40 is provided whose internal diameter fits the external diameter of the light-conducting rod 22. The sliding sleeve 40 consists of an elastomer and adheres to the light-conducting rod under a certain pre-stress. Due to its elasticity it can be pushed even beyond the curvature 42 of the light-conducting rod. Further on in FIG. 4 the layout is illustrated in a sectional view.

The sliding sleeve 40 carries two pivot pins 44 and 46 at the sides. The pivot pins are overlapped by supporting arms 48 and 50 of a mirror mounting 54 and can be pivoted thereat. The mirror mounting 54 carries the mirror 28 whose diameter is slightly larger than the diameter of the light-emitting surface 56 of the light-conducting rod 22. These elements offer a mirror attachment in this respect.

The mirror mounting 54 can be pivoted by pivoting about the pins 44 and 46 into a lateral position but also into the calibration position illustrated in the Figure. In this position, the mirror 28 lies flat against the light-emitting surface 56 which forms an alignment aid in this respect.

In operation, the sliding sleeve 40 is initially pushed to the front to such an extent that it is possible to pivot the mirror mounting 54. Then, it is again pushed back such that the mirror 28 is supported by the light-emitting surface 56. In this state the calibration mode is turned on and calibration is performed in the manner described above.

After the calibration process has taken place the sliding sleeve 40 is either pulled off without further ado, or it is pushed into the rear position illustrated in FIG. 3 in dotted lines, of course after the mirror mounting 54 has been pivoted into the lateral position.

In the rear position of the mirror mounting 54 and the sliding sleeve 40 the rest of the light-conducting rod 22 is exposed, and there is no obstruction during the light curing process in the mouth of the patient.

In a further embodiment the mirror mounting is combined with antidazzle flaps known per se which simultaneously make possible an antidazzle protection when the mirror mounting is pivoted to the outside.

In a further embodiment the mirror element is configured from an adhesive film which is adhered to the light-emitting surface 56 and discarded after it has been used.

In a further embodiment it is provided to shift the light source in the handpiece to the front end of a rod which then is not configured as a light-conducting rod but as a voltage supply and cooling rod. In this case, the related sensor 30 is also attached to the front end of the handpiece, namely next to the light source, preferably in a radial direction towards the outside thereof.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

What is claimed is:

1. A light curing device for curing dental materials, comprising
a handpiece (14) comprising a light source, a light-conducting rod (22), which is attached to the handpiece (14) in a replaceable and detachable manner, and at least one sensor (30) which is positioned in the handpiece (14);
a base station (12); and
a control device (18) with at least one curing program for polymerizing the dental material;
characterized in that a mirror (28) with a predetermined reflection parameter with a reflection value of more than 90% is provided at the base station (12) of the light curing device (10) or at the handpiece (14) of the light curing device (10), and in that the control device (18) comprises a calibration program which can be actuated automatically or by user intervention when the light curing device (10) with the front end (24) of the light-conducting rod (22) is directed towards the mirror (28).

2. The light curing device as claimed in claim 1, characterized in that the sensor (30) and the light source are disposed adjacent to a light input end of the light-conducting rod (22).

3. The light curing device as claimed in claim 2, characterized in that the light curing device (10) comprises an alignment aid by means of which an optical axis of a light output end of the light-conducting rod (22) can be aligned perpendicular relative to a reflection area of the mirror (28), with a deviation of less than 5°.

4. The light curing device as claimed in claim 3, characterized in that the calibration program adjusts a rated light power of the light source based on a measurement result of the sensor (30).

5. The light curing device as claimed in claim 4, characterized in that the mirror (28) is pivotably attached as a measurement mirror to an antidazzle device which is attached to the light output end of the light-conducting rod (22).

6. The light curing device as claimed in claim 5, characterized in that the mirror (28) is attached to a base station (12) of the light curing device (10) as a measurement mirror.

7. The light curing device as claimed in claim 6, characterized in that the base station (12) of the light curing device (10) comprises a setdown position for the handpiece (14) in which the handpiece (14) can be picked up in or at a predetermined original position, and in that the mirror (28) as a measurement mirror at the front end (24) of the light curing device (10) is aligned parallel to the light output end of the light-conducting rod (22) and spaced apart from it slightly by less than 5 mm.

8. The light curing device as claimed in claim 7, characterized in that the handpiece (14) comprises a presence sensor for the light-conducting rod (22) by means of which it can be determined if a light-conducting rod (22) is inserted at the handpiece (14) or not inserted, and in that the control device (18) automatically performs the self-calibration program or requests to have it performed, when the presence sensor has reported the change of the light-conducting rod (22) to the control device (18).

9. The light curing device as claimed in claim 8, characterized in that the control device (18) is connected to a display device which displays the operating state of the light curing device (10), comprising the execution of the calibration program, and points out to the user that the usual operational function of the light curing device (10) is disabled during the calibration process.

10. The light curing device as claimed in claim 9, characterized in that the display device indicates the light power which is measured by the optical sensor (30) and used for the calibration process as a numerical value.

11. The light curing device as claimed in claim 10, characterized in that the self-calibration program of the control device (18) proportionally prolongs the light curing cycle with a decreasing and accordingly lower measured power of the light sources by up to 20%, and in that the exposure time is prolonged automatically.

12. The light curing device as claimed in claim 11, characterized in that the alignment aid is configured as a sleeve which surrounds the mirror (28) and shields it laterally and extends in front of the mirror and comprises an internal diameter which matches the front end (24) of the light-conducting rod (22).

13. The light curing device as claimed in claim 12, characterized in that the mirror (28) can be attached to the front end (24) of the light-conducting rod (22) as a mirror element.

14. The light curing device as claimed in claim 13, characterized in that the mirror element is a film which can be adhered onto the front end (24) of the light-conducting rod (22) and which is optionally disposable.

15. A light curing device for curing dental materials, comprising
a handpiece (14) comprising a light source, and at least one sensor (30) which is positioned in the handpiece (14);
a base station (12); and
a control device (18) with at least one curing program for polymerizing the dental material;
characterized in that a mirror (28) with a predetermined reflection parameter with a reflection value of more than 90% is provided at the base station (12) of the light curing device (10) or at the handpiece (14) of the light curing device (10), and in that the control device (18) comprises a calibration program which can be actuated automatically or by user intervention when the light curing device (10) with the front end (24) of the handpiece (14) is directed towards the mirror (28).

16. The light curing device as claimed in claim 11, characterized in that the exposure time is prolonged automatically from 5 seconds to 6 seconds.
